(19) United States
(12) Patent Application Publication
(10) Pub. No.: US 2003/0232079 A1
(43) Pub. Date: Dec. 18, 2003

(54) DPC 333 formulation having unique biopharmaceutical characteristics

(76) Inventors: Irma Helene Benedek, Wilmington, DE (US); Michael James Fossler, Elkton, MD (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/389,525

(22) Filed: Mar. 14, 2003

Related U.S. Application Data
(60) Provisional application No. 60/366,944, filed on Mar. 22, 2002.
(60) Provisional application No. 60/400,198, filed on Aug. 1, 2002.

(51) Int. Cl. A61K 31/4709; A61K 9/22

(52) U.S. Cl. 424/468; 514/314

(57) ABSTRACT

Oral dosage forms of the crystalline, free base of ([1-(R)]-3-amino-N-hydroxy-alpha-(2-methylpropyl)-3-[4-[(2-methyl-4-quinolinyl)methoxy]phenyl]-2-oxo-1-pyrrolidineacetamide, referred to herein as DPC 333, having unique biopharmaceutical characteristics are provided. Also provided are methods for using these oral dosage forms to inhibit tumor necrosis factor-alpha convertase (TACE) and to treat inflammatory diseases characterized by TNFα overproduction.

### CROSS-REFERENCE TO RELATED APPLICATIONS

This application claims the priority benefits of U.S. Provisional Application No. 60/366,944, filed Mar. 22, 2002, and U.S. Provisional Application No. 60/400,198, filed Aug. 1, 2002, all of which are fully incorporated herein by reference.

### FIELD OF THE INVENTION

The present invention is directed to oral formulations of the crystalline, free-base form of [1-(R)]-3-amino-N-hydroxy-alpha-(2-methylpropyl)-3-[4-[(2-methyl-4-quinolinyl)methoxy]phenyl]-2-oxo-1-pyrrolidineacetamide, referred to herein as DPC 333, having unique biopharmaceutical characteristics which are useful as inhibitors of tumor necrosis factor-alpha convertase (TACE). Such inhibitors are useful in the treatment of inflammatory diseases characterized by TNFα overproduction.

### BACKGROUND OF THE INVENTION

DPC 333, ([1-(R)]-3-amino-N-hydroxy-alpha-(2-methylpropyl)-3-[4-[(2-methyl-4-quinolinyl)methoxy]phenyl]-2-oxo-1-pyrrolidineacetamide, or can also be named (2R)-2-((3R)-3-amino-3-{-[(2-methyl-4-quinolinyl)methoxy]phenyl}-2-oxopyrrolidinyl)-N-hydroxy-4-methylpentanamide), is a potent selective and orally active inhibitor of human tumor necrosis factor-alpha convertase (TACE), and may be used for the treatment of inflammatory diseases characterized by TNFα overproduction including, but not limited to rheumatoid arthritis. The structure of DPC 333 is depicted below as Formula (I):

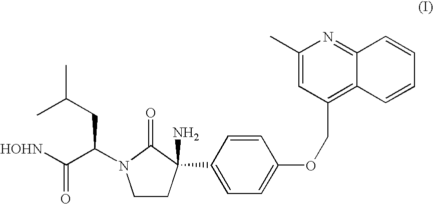

Methods for the synthesis of the bis-trifluoroacetic acid salt of DPC 333 are disclosed in U.S. Pat. No. 6,057,336, the teachings of which are incorporated herein by reference in their entirety.

In the present invention, the safety, tolerability, and pharmacokinetic characteristics of single ascending doses of an oral DPC formulation are provided as well as the effect of food on single dose pharmacokinetics of this oral DPC formulation.

### SUMMARY OF THE INVENTION

The present invention provides an oral dosage form of DPC 333 having a mean terminal disposition half-life (T_{1/2}) of about 2 hours to about 7 hours in single dose administrations ranging from 15 mg to 530 mg.

The present invention also provides an oral dosage form of DPC 333 exhibiting a greater than proportional increase in mean area under the plasma concentration versus time curve from time zero to time infinity (AUC) at a single dose greater than 225 mg.

The present invention further provides an oral dosage form of DPC 333 which, when administered with food exhibits a decreased AUC of about 23% and a decreased Cmax of about 40% as compared to administration of the oral dosage form of DPC 333 when administered in a fasted state.

In a preferred embodiment of the present invention the oral DPC dosage form provides:

a mean maximum plasma concentration (Cmax) of about 88 nM to about 180 nM, a mean time of maximum plasma concentration (Tmax) of about 0.5 hours to about 1.0 hours, or a mean area under the plasma concentration versus time curve from time zero to time infinity (AUC) of about 255 nM▪hour to about 430 nM▪hour when administered as a single dose of 15 mg;

a mean maximum plasma concentration (Cmax) of about 170 nM to about 435 nM, a mean time of maximum plasma concentration (Tmax) of about 0.5 hours to about 1.0 hours, or a mean area under the plasma concentration versus time curve from time zero to time infinity (AUC) of about 450 nM▪hour to about 900 nM▪hour when administered as a single dose of 25 mg;

a mean maximum plasma concentration (Cmax) of about 355 nM to about 655 μM, a mean time of maximum plasma concentration (Tmax) of about 0.25 hours to about 1.0 hours, or a mean area under the plasma concentration versus time curve from time zero to time infinity (AUC) of about 930 nM▪hour to about 1425 nM▪hour when administered as a single dose of 40 mg;

a mean maximum plasma concentration (Cmax) of about 965 nM to about 1870 μM, a mean time of maximum plasma concentration (Tmax) of about 0.5 hours to about 1.0 hours, or a mean area under the plasma concentration versus time curve from time zero to time infinity (AUC) of about 2245 nM▪hour to about 4160 nM▪hour when administered as a single dose of 80 mg;

a mean maximum plasma concentration (Cmax) of about 1535 nM to about 2345 nM, a mean time of maximum plasma concentration (Tmax) of about 0.5 hours to about 1.0 hours, or a mean area under the plasma concentration versus time curve from time zero to time infinity (AUC) of about 3335 nM▪hour to about 6210 nM▪hour when administered as a single dose of 120 mg;

a mean maximum plasma concentration (Cmax) of about 2625 nM to about 3650 μM, a mean time of maximum plasma concentration (Tmax) of about 0.5 hours to about 1.0 hours, or a mean area under the plasma concentration versus time curve from time zero to time infinity (AUC) of about 6380 nM▪hour to about 9400 nM▪hour when administered as a single dose of 225 mg;

a mean maximum plasma concentration (Cmax) of about 6585 nM to about 15420 nM, a mean time of maximum plasma concentration (Tmax) of about 0.25 hours to about 1.0 hours, or a mean area under the plasma concentration versus time curve from time zero to time infinity (AUC) of about 18375 nM▪hour to about 28820 nM▪hour when administered as a single dose of 345 mg; or

a mean maximum plasma concentration (Cmax) of about 9450 nM to about 18072 nM, a mean time of maximum plasma concentration (Tmax) of about 0.5 hours to about 1.0 hours, or a mean area under the plasma concentration versus time curve from time zero to time infinity (AUC) of about 22040 nM▪hour to about 39480 nM▪hour when administered as a single dose of 530 mg.

The present invention also provides a method of inhibiting human tumor necrosis factor-alpha convertase (TACE) comprising administering to a mammal one of the above-described oral dosage forms of DPC 333.

The present invention also provides a method of treating inflammatory diseases characterized by TNFα overproduction comprising administering to a mammal one of the above-described oral dosage forms of DPC 333.

The present invention also provides a method of treating a condition or disease mediated by MMPs, TACE, or a combination thereof in a mammal, comprising: administering to the mammal in need of such treatment one of the above-described oral dosage forms of DPC 333.

The present invention provides a method comprising: administering one of the above-described oral dosage forms of DPC 333 to treat a condition or disease mediated by MMPs, TACE, or a combination thereof.

### BRIEF DESCRIPTION OF THE FIGURES

FIG. 1 shows the mean DPC 333 plasma concentration versus time curves after administration to subjects in a single dose ranging from 15 to 530 mg. The 15 mg single dose of DPC 333 is represented by a filled square; the 25 mg single dose of DPC 333 is represented by a filled triangle; the 40 mg dose of DPC 333 is represented by an inverted filled triangle; the 80 mg dose of DPC 333 is represented by a filled diamond; the 120 mg dose of DPC 333 is represented by a filled circle; the 225 mg dose of DPC 333 is represented by an open square; the 345 mg dose of DPC 333 is represented by an open triangle; and the 530 mg dose of DPC 333 is represented by an inverted open triangle.

FIG. 2 shows mean and individual values for DPC 333 AUC (0-inf) for doses of DPC ranging from 15 to 530 mg. Mean values are depicted by open diamonds and individual values are depicted by filled triangles.

### DETAILED DESCRIPTION OF THE INVENTION

The present invention provides oral dosage formulations (also referred to herein as dosage forms) comprising DPC 333, (the crystalline, free-base form of [1-(R)]-3-amino-N-hydroxy-alpha-(2-methylpropyl)-3-[4-[(2-methyl-4-quinolinyl)methoxy]phenyl]-2-oxo-1-pyrrolidineacetamide), that are useful as potent selective and orally active inhibitors of human tumor necrosis factor-alpha convertase (TACE). The dosage forms of the present invention may be used in the treatment of inflammatory diseases characterized by TNFα overproduction including, but not limited to rheumatoid arthritis.

In particular, the present invention relates to orally administered solutions or suspensions comprising DPC 333 that have unique biopharmaceutical characteristics.

The active ingredient of the oral dosage forms of the present invention is DPC 333 and is depicted below as Formula (I):

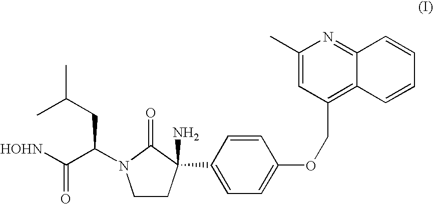

Methods for the synthesis of DPC 333 are disclosed in U.S. Pat. No. 6,057,336, the teachings of which are incorporated herein by reference in their entirety.

In the present invention, oral formulations are provided comprising a therapeutically effective amount of DPC 333. In a preferred embodiment, the oral formulation is prepared as a solution or suspension a few hours, preferably less than 2 hours, prior to administration. In this embodiment, DCP is provided as a powder that is dissolved prior to administration in a suitable liquid formulation. While various liquid formulations well known in the art may be used, a preferred liquid formulation comprises a small volume, preferably 2 milliliters or less, of a wetting solution such as a 0.5% Pluronic® F68 stock solution which is added first to the DPC 333 powder and swirled so that all the powder is wetted. A larger volume, preferably 18 to 58 milliliters of simple syrup:distilled water solution is then added to the bottle and the bottle is shaken vigorously to mix. Depending upon the concentration of DPC 333 either a solution or suspension will result.

The safety, tolerability, and pharmacokinetics of an oral dose form of DPC 333 were evaluated in a double-blind, placebo controlled, ascending single dose study in healthy male and female human subjects. Subjects in each cohort were randomized to receive either a single dose of DPC 333 (n=6) or placebo (n=2). Doses examined were 15, 25, 40, 80, 120, 225, 345, and 530 mg. Safety measurements included monitoring of AEs, standard clinical laboratory tests, ECGs, and vital sign measurements.

Blood samples were obtained pre-dose (0 hour) and 0.25, 0.5, 1, 1.5, 2, 2.5, 3, 4, 6, 8, 12, 18, 24, 36 and 48 hours post-dose administration. DPC 333 was quantified plasma extracts from these blood samples by a solid phase method followed by turbo ion spray liquid chromatography employing tandem mass spectroscopic detection (LC/MS/MS). The validated assay range for DPC 333 using 0.2 mL of plasma was 1.05 to 2098 nM. The assay was specific for DPC 333.

The following pharmacokinetic parameters for DPC 333 were assessed following a single dose administration of the DPC 333 oral dosage form to a subject: Cmax, Tmax, AUC, T_{1/2}, Clo, and C₁₂. Cmax is defined as the observed maximum plasma concentration. Tmax is defined as the time of observed maximum plasma concentration. AUC is defined as the area under the plasma concentration-time curve from time zero to time infinity; T_{1/2} is defined as the terminal disposition half-life. Clo is defined as the apparent oral clearance; calculated as dose/AUC; and C₁₂ (plasma concentration at 12 hours).

The following table provides pharmacokinetic data for DPC 333 in this single dose study.

TABLE 1
Single Dose Pharmacokinetics of DPC 333 in Healthy Volunteers

| | | 15 mg | 25 mg | 40 mg | 80 mg | 120 mg | 225 mg | 345 mg | 530 mg |
|---|---|---|---|---|---|---|---|---|---|
| Parameter | Statistic | (n = 6) | (n = 6) | (n = 6) | (n = 12) | (n = 6) | (n = 6) | (n = 6) | (n = 6) |
| AUC (0-∞) | Mean | 341.9 | 675.9 | 1179.0 | 3202.2 | 4773.6 | 7888.4 | 23598.4 | 30762.2 |
| (nM · h) | SD | 86.3 | 223.1 | 244.6 | 957.1 | 1436.5 | 1507.8 | 5220.5 | 8718.5 |
| | CV % | 25.2 | 33.0 | 20.7 | 29.9 | 30.1 | 19.1 | 22.1 | 28.3 |
| CLo | Mean | 98.0 | 85.7 | 74.5 | 57.3 | 56.0 | 61.6 | 32.0 | 38.6 |
| (L/hr) | SD | 28.5 | 31.4 | 19.6 | 18.7 | 13.2 | 11.1 | 7.04 | 10.8 |
| | CV % | 29.1 | 36.6 | 26.3 | 32.7 | 23.6 | 18.0 | 22.0 | 28.0 |
| Cmax | Mean | 133.6 | 303.2 | 505.0 | 1420.3 | 1941.5 | 3139.5 | 11001.7 | 13761.0 |
| (nM) | SD | 45.1 | 132.0 | 148.8 | 450.5 | 404.7 | 510.8 | 4415.5 | 4310.8 |
| | CV % | 33.7 | 43.5 | 29.5 | 31.7 | 20.8 | 16.3 | 40.1 | 31.3 |
| Tmax | Median | 0.75 | 0.5 | 0.5 | 0.5 | 0.5 | 0.75 | 0.75 | 0.5 |
| (hrs) | Range | (0.5-1.0) | (0.5-1.0) | (0.25-1.0) | (0.5-1.0) | (0.5-1.0) | (0.5-1.0) | (0.25-1.0) | (0.5-1.0) |
| T½ | Mean^{a} | 2.18 | 2.86 | 3.41 | 4.05 | 4.89 | 5.53 | 6.39 | 6.04 |
| (hrs) | SD^{a} | 0.21 | 0.86 | 0.67 | 0.51 | 0.99 | 0.88 | 0.73 | 0.58 |
| | CV % | 9.5 | 30.2 | 19.6 | 12.6 | 20.3 | 16.0 | 11.4 | 9.6 |
| C₁₂ | Mean | 2.09 | 4.41 | 6.36 | 20.4 | 30.26 | 49.03 | 152.8 | 167.3 |
| (nM) | SD | 0.79 | 2.08 | 1.03 | 6.60 | 12.74 | 9.32 | 32.2 | 43.9 |
| | CV % | 37.9 | 47.2 | 16.3 | 32.3 | 42.1 | 19.01 | 21.1 | 26.3 |

^{a}Data are harmonic mean ± pseudo-standard deviation

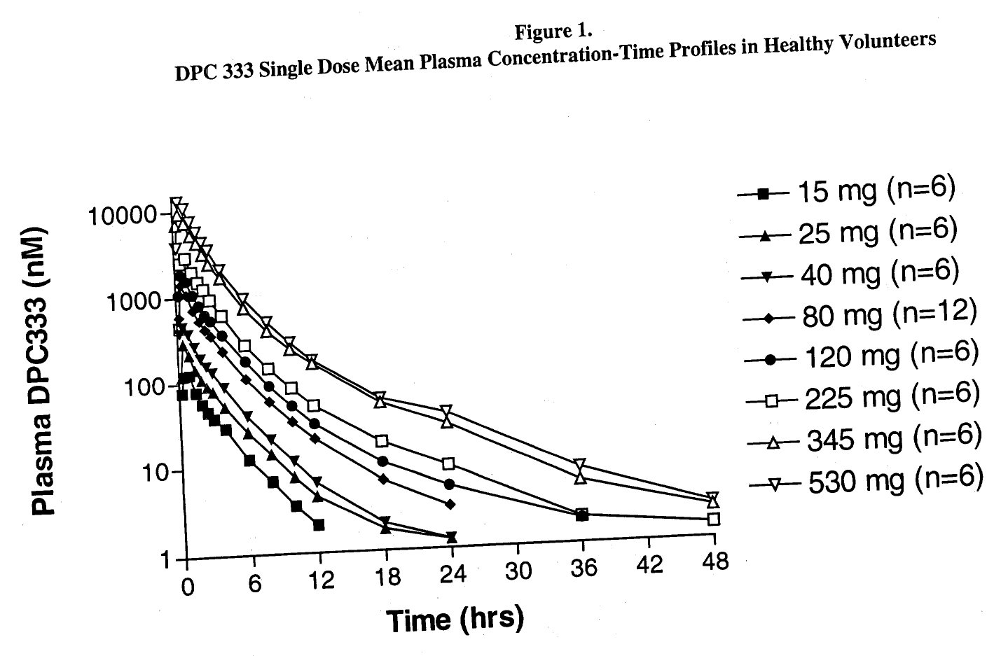

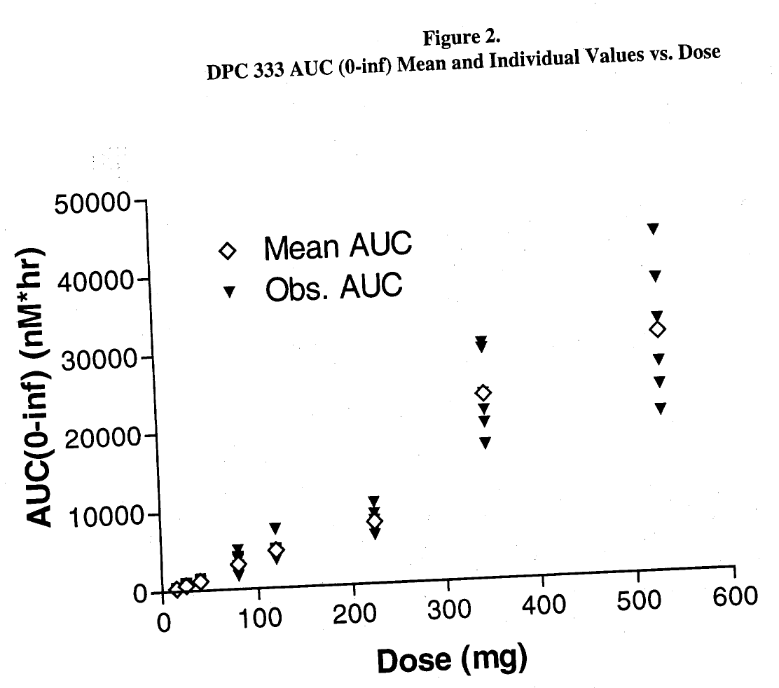

As can bee seen, upon oral administration, DPC 333 is rapidly and well absorbed with Tmax values of less than 1 hour post dose (see Table 1 and FIGS. 1 and 2). The mean half-life estimates for the lower doses (≦80 mg) are relatively short and gradually increase with dose to about 6 hours for the higher doses. A greater-than-proportional increase in exposure (AUC) was observed at the 345 mg and 530 mg doses.

The effect of food on the safety, tolerability and pharmacokinetics of this oral dose form of DPC 333 was also examined in a randomized, open label, two period balanced crossover study in one cohort of male subjects. Each subject received a single oral 225 mg dose of DPC 333 on two occasions administered at least one week apart. On one occasion the dose was given in the fasted state and on the other occasion the dose was administered after a standard, high-fat breakfast meal. The following table provides pharmacokinetic data from this study.

TABLE 2
DPC 333 Pharmacokinetics after Fasted and Fed Administration

| | Fasted (n = 6) | Fed^{a} (n = 6) | Geometric Mean |
|---|---|---|---|
| Parameter | 225 mg | 225 mg | Ratio^{b} (90% CI) |
| AUC (0-∞), nM · h | 12454 ± 2790 | 9493 ± 1711 | 0.767 |
| | | | (0.715-0.824) |
| AUC (0-t), nM · h | 12442 ± 2789 | 9477 ± 1710 | 0.767 |
| | | | (0.714-0.824) |
| Cmax, nM | 5703 ± 1425 | 3412 ± 832 | 0.597 |
| | | | (0.450-0.792) |
| Tmax^{c}, hrs | 0.5 | 0.5 | NA |
| | (0.5-1.5) | (0.28-1.0) | |

^{a}Meal consisted of 2 eggs fried in oil, 2 strips of bacon, 2 slices of toast with butter, 4 ounces of hash brown potatoes, and 8 ounces of whole milk (approximately 1000 Calories [1000 Kcal] of which approximately 50% were from fat)
^{b}Using fasted treatment as the reference
^{c}Data are median (range)
CI = Confidence interval; NA = Not applicable
Note:
Data are mean ± standard deviation unless otherwise noted.

Thus, as shown, administration of DPC 333 with food decreased both the AUC (about 23%) and Cmax (about 40%) as compared to DPC 333 when administered in the fasted state.

Differences in pharmacokinetics of DPC 333 based upon gender were also examined. The following table provides a comparison of DPC 333 pharmacokinetics by gender in fasted subjects receiving a single dose of 225 mg of DPC 333.

TABLE 3
Comparison of DPC 333 Pharmacokinetics by Gender

| | Males (n = 6) | Females (n = 6) | Males (n = 6) |
|---|---|---|---|
| | Part 1: 225 mg | Part 1: 225 mg | Part 2: 225 mg |
| Parameter | (fasted) | (fasted) | (fasted) |
| AUC (0-∞) | 7888 ± 1508 | 11355 ± 2174 | 12454 ± 2790 |
| nM · h | | | |
| Cmax, nM | 3140 ± 511 | 6649 ± 1752 | 5703 ± 1425 |
| Tmax^{a}, hrs | 0.75 | 0.51 | 0.50 |
| | (0.5-1.0) | (0.5-1.0) | (0.5-1.5) |
| T1/2^{b}, hrs | 5.53 ± 0.88 | 5.93 ± 0.66 | 6.19 ± 1.21 |

^{a}Data are median (range)
^{b}Data are harmonic mean ± pseudo-standard deviation
Note:
Data mean ± standard deviation unless otherwise noted

No apparent differences in the pharmacokinetics of DPC 333 due to gender were observed.

For all safety parameters measured there were no dose-related mean trends observed in the doses evaluated. There were no discontinuations because of an AE nor any serious AEs reported and the criteria for an intolerable dose were not achieved for the doses evaluated.

Thus, as demonstrated by these studies, the oral dose form of DPC 333 of the present invention is rapidly and well absorbed after oral dosing. Further, this oral dose form is well tolerates in healthy volunteers.

Accordingly, the present invention provides an oral dosage form of DPC 333 having a mean terminal disposition half-life (T_{1/2}) of about 2 hours to about 7 hours in single dose administrations ranging from 15 mg to 530 mg.

The present invention also provides an oral dosage form of DPC 333 exhibiting a greater than proportional increase in mean area under the plasma concentration versus time curve from time zero to time infinity (AUC) at a single dose greater than 225 mg.

In a preferred embodiment of the present invention the oral DPC dosage form provides:

a mean maximum plasma concentration (Cmax) of about 88 nM to about 180 nM, a mean time of maximum plasma concentration (Tmax) of about 0.5 hours to about 1.0 hours, or a mean area under the plasma concentration versus time curve from time zero to time infinity (AUC) of about 255 nM▪hour to about 430 nM▪hour when administered as a single dose of 15 mg;

a mean maximum plasma concentration (Cmax) of about 170 nM to about 435 nM, a mean time of maximum plasma concentration (Tmax) of about 0.5 hours to about 1.0 hours, or a mean area under the plasma concentration versus time curve from time zero to time infinity (AUC) of about 450 nM▪hour to about 900 nM▪hour when administered as a single dose of 25 mg;

a mean maximum plasma concentration (Cmax) of about 355 nM to about 655 μM, a mean time of maximum plasma concentration (Tmax) of about 0.25 hours to about 1.0 hours, or a mean area under the plasma concentration versus time curve from time zero to time infinity (AUC) of about 930 nM▪hour to about 1425 nM▪hour when administered as a single dose of 40 mg;

a mean maximum plasma concentration (Cmax) of about 965 nM to about 1870 μM, a mean time of maximum plasma concentration (Tmax) of about 0.5 hours to about 1.0 hours, or a mean area under the plasma concentration versus time curve from time zero to time infinity (AUC) of about 2245 nM▪hour to about 4160 nM▪hour when administered as a single dose of 80 mg;

a mean maximum plasma concentration (Cmax) of about 1535 nM to about 2345 nM, a mean time of maximum plasma concentration (Tmax) of about 0.5 hours to about 1.0 hours, or a mean area under the plasma concentration versus time curve from time zero to time infinity (AUC) of about 3335 nM▪hour to about 6210 nM▪hour when administered as a single dose of 120 mg;

a mean maximum plasma concentration (Cmax) of about 2625 nM to about 3650 μM, a mean time of maximum plasma concentration (Tmax) of about 0.5 hours to about 1.0 hours, or a mean area under the plasma concentration versus time curve from time zero to time infinity (AUC) of about 6380 nM▪hour to about 9400 nM▪hour when administered as a single dose of 225 mg;

a mean maximum plasma concentration (Cmax) of about 6585 nM to about 15420 nM, a mean time of maximum plasma concentration (Tmax) of about 0.25 hours to about 1.0 hours, or a mean area under the plasma concentration versus time curve from time zero to time infinity (AUC) of about 18375 nM▪hour to about 28820 nM▪hour when administered as a single dose of 345 mg; or

a mean maximum plasma concentration (Cmax) of about 9450 nM to about 18072 nM, a mean time of maximum plasma concentration (Tmax) of about 0.5 hours to about 1.0 hours, or a mean area under the plasma concentration versus time curve from time zero to time infinity (AUC) of about 22040 nM▪hour to about 39480 nM▪hour when administered as a single dose of 530 mg.

The present invention further provides an oral dosage form of DPC 333 which, when administered with food exhibits a decreased AUC of about 23% and a decreased Cmax of about 40% as compared to administration of the oral dosage form of DPC 333 when administered in a fasted state.

Also provided in the present invention are kits comprising packaging including one or more bottles of powdered DPC 333, a wetting solution such 0.5% Pluronic® F68 stock solution and a simple syrup:distilled water solution. Also provided in the kits are package inserts or labels indicating to a user how to formulate the oral DPC 333 dosage form and that DPC 333 may be suitable for the treatment of disease characterized by overproduction of TNFα. Kits of the present invention may further comprise a package insert or label warning the user of potential side effects, adverse reactions or drug interactions.

The present invention also provides methods for inhibiting human tumor necrosis factor-alpha convertase (TACE) comprising administering to a mammal one of the above-described oral dosage forms of DPC 333.

The present invention also provides a method of treating inflammatory diseases characterized by TNFα overproduction comprising administering to a mammal one of the above-described oral dosage forms of DPC 333.

The present invention also provides a novel method of treating a condition or disease mediated by MMPs, TACE, aggrecanase, or a combination thereof in a mammal, comprising: administering to the mammal in need of such treatment one of the above-described oral dosage forms of DPC 333.

The present invention also provides a novel method comprising: administering one of the above-described oral dosage forms of DPC 333 to treat a condition or disease mediated by MMPs, TACE, aggrecanase, or a combination thereof.

The present invention also provides a novel method of treating a disease or condition, wherein the disease or condition is selected from acute infection, acute phase response, age related macular degeneration, alcoholic liver disease, allergy, allergic asthma, anorexia, aneurism, aortic aneurism, asthma, atherosclerosis, atopic dermatitis, autoimmune disease, autoimmune hepatitis, Bechet's disease, cachexia, calcium pyrophosphate dihydrate deposition disease, cardiovascular effects, chronic fatigue syndrome, chronic obstruction pulmonary disease, coagulation, congestive heart failure, corneal ulceration, Crohn's disease, enteropathic arthropathy, Felty's syndrome, fever, fibromyalgia syndrome, fibrotic disease, gingivitis, glucocorticoid withdrawal syndrome, gout, graft versus host disease, hemorrhage, HIV infection, hyperoxic alveolar injury, infectious arthritis, inflammation, intermittent hydrarthrosis, Lyme disease, meningitis, multiple sclerosis, myasthenia gravis, mycobacterial infection, neovascular glaucoma, osteoarthritis, pelvic inflammatory disease, periodontitis, polymyositis/dermatomyositis, post-ischaemic reperfusion injury, post-radiation asthenia, psoriasis, psoriatic arthritis, pulmonary emphysema, pydoderma gangrenosum, relapsing polychondritis, Reiter's syndrome, rheumatic fever, rheumatoid arthritis, sarcoidosis, scleroderma, sepsis syndrome, Still's disease, shock, Sjogren's syndrome, skin inflammatory diseases, solid tumor growth and tumor invasion by secondary metastases, spondylitis, stroke, systemic lupus erythematosus, ulcerative colitis, uveitis, vasculitis, and Wegener's granulomatosis. The present invention also provides a method for treating inflammatory disorders, comprising: administering, to a host in need of such treatment, one of the above-described oral dosage forms of DPC 333, in combination with one or more additional anti-inflammatory agents selected from selective COX-2 inhibitors, interleukin-1 antagonists, dihydroorotate synthase inhibitors, p38 MAP kinase inhibitors, TNF-α inhibitors, TNF-α sequestration agents, and methotrexate.

The present invention also provides a novel article of manufacture, comprising:

(a) a first container;

(b) a pharmaceutical composition located within the first container, wherein the composition, comprises: a first therapeutic agent, comprising: one of the above-described oral dosage forms of DPC 333; and,

(c) a package insert stating that the pharmaceutical composition can be used for the treatment of an inflammatory disorder.

The present invention also provides a novel article of manufacture, comprising:

(a) a first container;

(b) a pharmaceutical composition located within the first container, wherein the composition, comprises: a first therapeutic agent, comprising: one of the above-described oral dosage forms of DPC 333; and,

(c) a package insert stating that the pharmaceutical composition can be used in combination with a second therapeutic agent to treat an inflammatory disorder.

The present invention also provides a novel article of manufacture, further comprising:

(d) a second container;

wherein components (a) and (b) are located within the second container and component (c) is located within or outside of the second container.

The following nonlimiting examples are provided to further illustrate the present invention.

### EXAMPLES

### Example 1

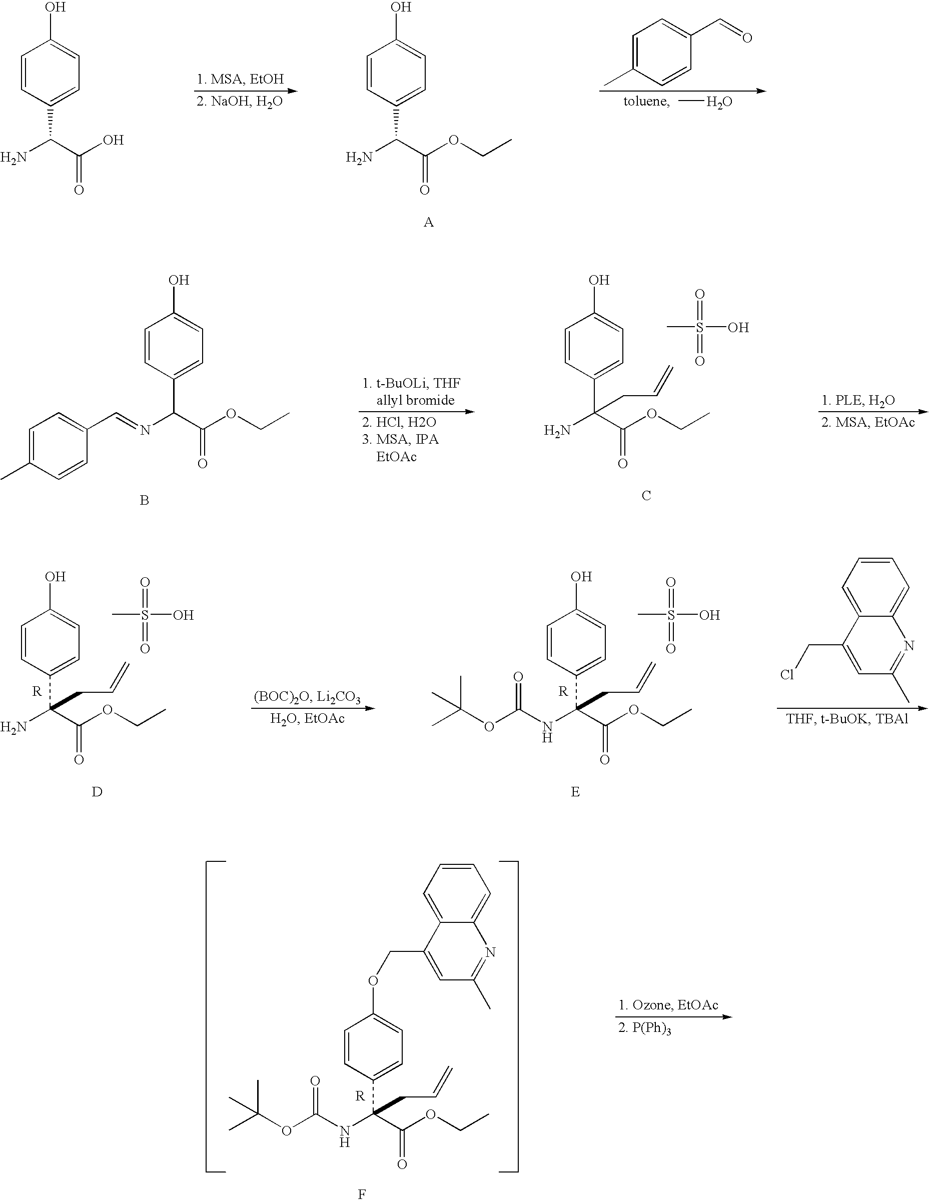

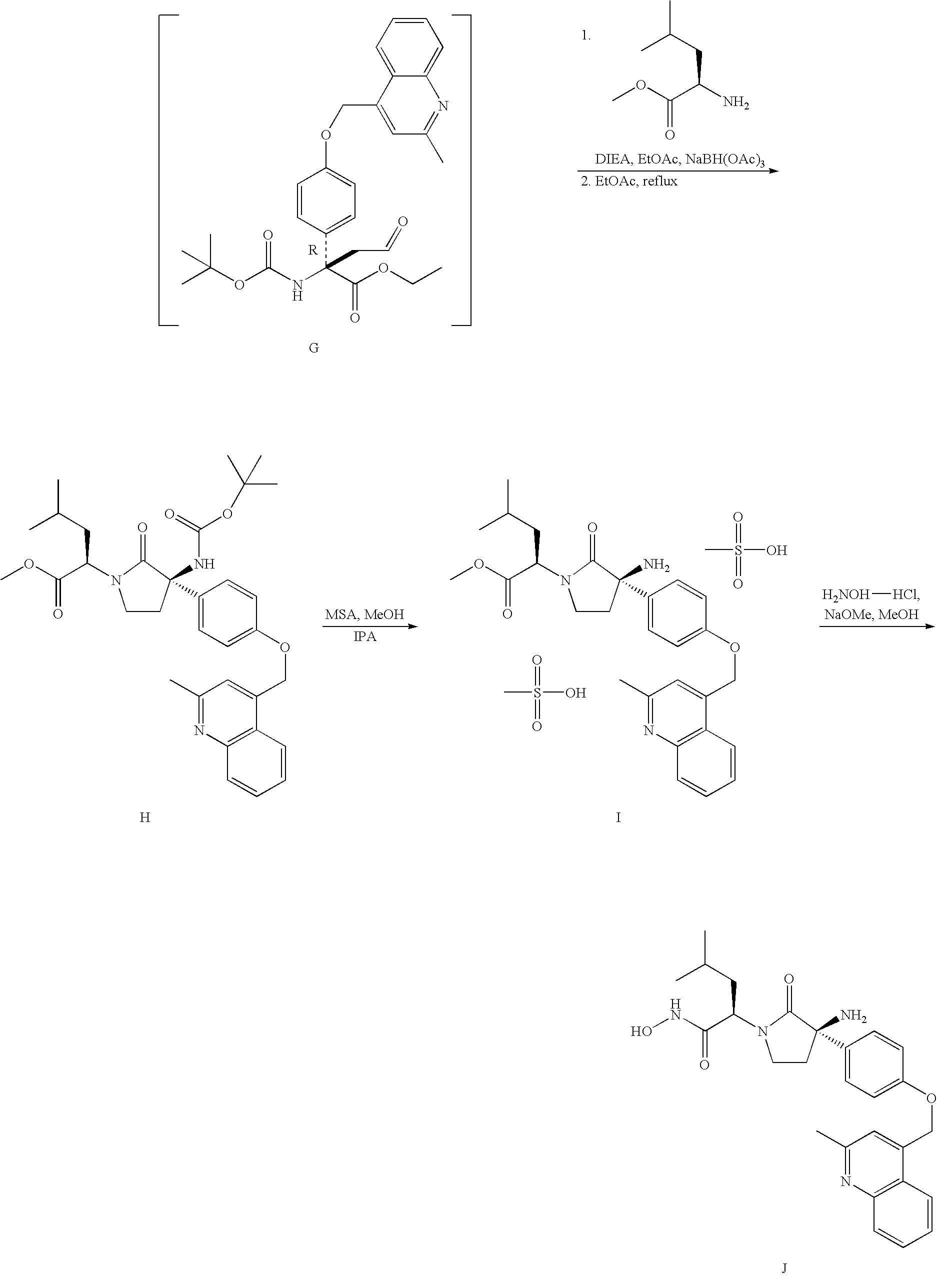

Preparation of Compound A

A 300-gallon reactor was charged with absolute ethyl alcohol (123 kg) and cooled to 10° C. D-4-hydroxyphenylglycine (78 kg, 0.47 kmol) was added, followed by methanesulfonic acid (86 kg, 0.89 kmol, 1.93 eq) at ≦78° C. The reaction mass was heated to 78° C. and aged for 2 h. The reactor was cooled to 55° C. and the reaction was determined complete by HPLC. The reactor was cooled to 40° C. and H₂O (468 L) was added. The reactor was cooled to 5° C. and 17% aqueous sodium hydroxide (prepared from 142 L H₂O, 71.3 kg 50% sodium hydroxide {0.89 kmol, 1.93 eq) was slowly added over 2 hours to provide a slurry of Compound A at pH=7.0 to 7.5. The slurry was stirred at 5° C. for 1 hour. Compound A was isolated by centrifugation and washed with H₂O (3×106 kg). Compound A was vacuum dried at 50 to 55° C. to a constant weight. Compound A, 83.5 kg (92% yield), was isolated as a white to light yellow crystalline solid. ¹H NMR (400 MHz, DMSO-d₆) δ7.18 (2H, d, J=8.5 Hz), 6.73 (2H, d, J=8.6 Hz), 4.40 (1H, s), 4.15-3.95 (2H, m), 1.12 (3H, t, J=7.1 Hz). ¹³C NMR (100 MHz, DMSO-d₆) δ174.7, 157.1, 131.7, 128.2, 115.4, 60.6, 58.0, 14.3. Analysis Calculated for C₁₀H₁₃NO₃: C, 61.53; H, 6.71; N, 7.18. Found: C, 61.54; H, 6.57; N, 7.11.

Preparation of Compound B

A 300 gallon reactor was charged with toluene (685 kg) and cooled to 0° C. Compound A (80 kg, 0.41 kmol) was added followed by p-tolualdehyde (51.4 kg, 0.43 kmol, 1.04 eq) with a toluene (7 kg) flush. The reaction mass was distilled (90 to 115° C.) until approximately 180 kg distillate was collected. Fresh toluene, equivalent in mass to the distillate was added. The reaction mass was cooled to 85° C. and aged for 1 h, then cooled to 20° C. over 1 hour and aged for 2 h. Compound B was isolated by centrifugation and washed with a toluene (200 kg)-heptane (157 kg) mixture in three portions. Compound B was vacuum dried at 50 to 55° C. to a constant weight. Compound B, 118 kg (97% yield) was isolated as a white to light yellow crystalline solid. ¹H NMR (400 MHz, CDCl₃) δ8.25 (1H, s), 7.67 (2H, d, J=8.1 Hz), 7.26 (2H, d, J=8.6 Hz), 7.16 (2H, d, J=8.0 Hz), 6.76 (2H, d, J=8.6 Hz), 5.13 (1H, s), 4.25-4.11 (2H, m), 2.33 (3H, s), 1.20 (3H, t, J=7.1 Hz). ¹³C NMR (100 MHz, CDCl₃) δ172.0, 164.0, 156.2, 141.8, 132.7, 129.3, 129.2, 129.1, 128.8, 115.9, 75.7, 61.6, 21.5, 14.0.

Preparation of Compound C

A 300-gallon glass lined reactor was charged with tetrahydrofuran (215.5 kg, THF) and cooled to −5° C. Compound B (62.0 kg, 0.21 kmol) was added, followed by allyl bromide (26.7 kg, 0.22 kmol, 1.06 eq) with a THF (2.5 kg) chase. The reactor was cooled to −5° C. and charged with 2 M lithium tert-butoxide in THF (192.6 kg, 0.44 kmol, 2.1 eq) over 1 h at ≦5° C. The reaction mass was sampled for conversion after 30 min and determined complete by HPLC. The reaction was quenched by adding 2 M aqueous hydrochloric acid (326 kg, 0.625 kmol, 3 eq), pH=1 after quench. Heptanes (107 kg) were added and the layers were separated, retaining the product rich aqueous phase. The pH of the aqueous phase was adjusted to 8-9 by adding a 16% aqueous sodium hydroxide solution (prepared by combining 32 kg 50% aqueous sodium hydroxide {0.40 kmol, 1.9 eq} and 62 kg H₂O). Sodium chloride (31 kg) and ethyl acetate (369 kg) were added and the layers were separated, retaining the product rich organic phase. Solvent exchanged to ethyl acetate to a final volume of ˜250 L by distillation at atmospheric pressure. Ethyl acetate was added to adjust the final volume. Cooled to 60° C. and added 2-propyl alcohol (9.6 kg). Cooled to 50° C. and added methanesulfonic acid (20.1 kg, 0.21 kmol, 1 eq). Cooled to 20° C. over 2 hours. Cooled to 0° C. and aged for 1 hour. Compound C was isolated by centrifugation and washed with ethyl acetate (2×56 kg). Compound C was vacuum dried at 50 to 55° C. to a constant weight. Compound C, 57 kg (82% yield) was isolated as a white to light yellow crystalline solid. ¹H NMR (400 MHz, DMSO d₆) δ9.90 (1H, s), 8.83 (3H, s), 7.29 (2H, d, J=8.8 Hz), 6.85 (2H, d, J=8.8 Hz), 5.83-5.71 (1H, m), 5.30 (1H, d, J=17.0 Hz), 5.24 (1H, d, J=10.1 Hz), 4.30-4.15 (2H, m), 2.97 (2H, dd, J=6.8, 14.5 Hz), 2.35 (3H, s), 1.18 (3H t, J=7.1 Hz). ¹³C NMR (100 MHz, CD₃OD) δ171.4, 160.4, 131.1, 128.9, 127.1, 123.7, 117.4, 66.1, 64.7, 41.5, 40.0, 14.7.

Preparation of Compound D

Charged 120 L of water to a 100 gallon reactor. Heated to 40° C. Charged 12 kg of Compound C. Stirred at moderate rate to dissolve the mixture. Charged 0.73 kg of tris(hydroxymethyl)aminomethane. Charged 0.6 kg of Antarox BL-225. Carefully added 5 kg of NaOH (6 N, prepared earlier) while monitoring pH. The final pH was in the range of 6.8 to 7.5. Added NaOH (6 N) in small increments to adjust final pH to a range of 7.8 to 8.1. If pH was over 8.1, added HCl (6 N) to bring pH back to 8.0. Charged 0.27 kg PLE. Agitated the mixture at 150 rpm at 40° C. and pH=8.0±0.3 (NaOH (6 N) used for pH adjustment) for 5 h, at which time the reaction was judged complete by HPLC analysis. Cooled the reaction mass to 20° C. Charged 4 L of water. Charged 108 kg of ethyl acetate. Charged 18 kg of Celite®-560 and filtered through a Dacron® cloth on a Nutsche filter. Rinsed the reactor with 54 kg of ethylacetate and used the rinse to wash the filter-cake. Combined the wash with filtrate. Rinsed the reactor with 40 L of water and used the rinse to wash the filter-cake. Combined the wash with filtrate. Charged the combined filtrate/washes to the reactor and separated the layers, retaining both phases. Charged the aqueous layer back to the reactor and back extracted with 54 kg of ethyl acetate. Charged both the organic phases to the reactor. Washed combined organic layers with 40 kg of saturated aqueous sodium chloride solution. Separated aqueous (lower) layer. Distilled off ethyl acetate and residual water to a constant b.p. (77° C.±1° C.). Cooled the batch to ˜50° C. Charged 2 kg of 2-propyl alcohol. Charged 1.8 kg of methanesulfonic acid in 4 equal portions of ˜0.45 kg over a period of 20 min maintaining the temperature between 50 to 55° C. Cooled the batch to 25° C. over ˜100 min. Aged the slurry of crystalline Compound D MSA salt at 25° C. for 2 h. Cooled to 10° C. over ˜1 h. Filtered the slurry at 10° C. Rinsed the reactor with 12 kg of ethyl acetate twice and used the rinses to wash the filter-cake. Dried the product under vacuum at 50 to 55° C. with nitrogen purge to constant weight, 5 kg (42% yield). ¹H NMR (400 MHz, DMSO d₆) δ9.90 (1H, s), 8.83 (3H, s), 7.29 (2H, d, J=8.8 Hz), 6.85 (2H, d, J=8.8 Hz), 5.83-5.71 (1H, m), 5.30 (1H, d, J=17.0 Hz), 5.24 (1H, d, J=10.1 Hz), 4.30-4.15 (2H, m), 2.97 (2H, dd, J=6.8, 14.5 Hz), 2.35 (3H, s), 1.18 (3H t, J=7.1 Hz). ¹³C NMR (100 MHz, CD₃OD) δ171.4, 160.4, 131.1, 128.9, 127.1, 123.7, 117.4, 66.1, 64.7, 41.5, 40.0, 14.7. Analysis Calculated for C₁₄H₂₁NO₆S: C, 50.74; H, 6.39; N, 4.23; S, 9.68. Found: C, 50.43; H, 6.06; N, 4.08; S, 9.74.

Preparation of Compound E

A 300 gallon glass lined reactor was charged sequentially with Compound D (55 kg, 0.17 kmol), lithium carbonate (26.8 kg, 0.36 kmol, 2.2 eq), ethyl acetate (150 kg), di-tert-butyl dicarbonate ((BOC)₂O, 72.4 kg, 0.33 kmol, 1.95 eq), and water (269 L). Heated to 40±2° C. and aged for 14 hours. The reaction was sampled and determined complete by HPLC. Cooled the reaction mass to 20° C. and slowly charged acetic acid (32.9 kg, 0.55 kmol, 3.2 eq). Checked the pH, adjusted the pH to <6.5 with acetic acid. Separated the phases, retained the product rich organic phase. Washed the organic phase with water (269 L). Solvent exchanged to heptane to a final volume of 400 L by distillation at 100 mm Hg. The resulting slurry was cooled to 20° C. Compound E was isolated by filtration on a Nutsche filter and washed with heptane (2×75 kg). Compound E was vacuum dried at 50 to 55° C. to a constant weight. Compound E, 43 kg (78% yield) was isolated as a white to light yellow crystalline solid. ¹H NMR (400 MHz, CDCl₃) δ7.21 (2H, d, J=8.7 Hz), 6.57 (2H, d, J=7.7 Hz), 6.19 (1H, s), 5.75-5.58 (1H, m), 5.18 (d, 1H, J=14.0 Hz), 5.14 (d, 1H, J=8.2), 4.25-4.00 (2H, m), 3.48-3.30 (1H, m), 3.25-3.10 (1H, m), 1.44 (9H, s br), 1.16 (3H, t, J=14.2 Hz). ¹³C NMR (100 MHz, CDCl₃) δ172.5, 155.7, 154.0, 132.5, 130.6, 127.0, 119.4, 115.5, 79.9, 64.4, 62.2, 38.1, 28.4, 14.0. Analysis Calculated for C₁₈H₂₅NO₅: C, 64.46; H, 7.51; N, 4.18. Found: C, 60.03; H, 7.38; N, 4.14.

Preparation of Compound F

A reactor was charged with 120 kg compound E (0.36 kmol) and 532 kg THF and cooled to 5° C. A solution of 48.2 kg potassium t-butoxide (0.43 kmol, 1.20 eq.) and 150 kg THF was added while maintaining the temperature below 10° C., with a chase of 32 kg THF. Aged at 0 to 10° C. for 15 minutes then added 68.5 kg 4-chloromethyl-2-methylquinoline (AB9871, 0.36 kmol, 1.00 eq) and 6.6 kg tetrabutylammonium iodide (0.018 kmol, 0.05 eq). Heated to 35 to 40° C. for 3 hours at which time the reaction was judged complete by HPLC. Cooled to ˜20° C. Added 725 L water, 18.2 kg acetic acid (0.30 kmol, 0.85 eq) and 621 kg ethyl acetate and separated the phases. Washed the organic phase with a solution of 98 kg citric acid (0.51 kmol, 1.43 eq) and 900 L water. Washed the organic phase with a solution of 72 kg sodium chloride and 650 L water. To the organic phase added 690 L water and adjusted to pH 7.1 by addition of 33 L of 33 wt % aqueous NaOH. After the pH adjustment, the layers were separated and the aqueous layer was discarded. Washed the organic phase with a solution of 72 kg sodium chloride and 650 L water. Vacuum-distilled to ˜600 L. Portion-wise added an additional 1200 L ethyl acetate while continuing to vacuum distill at ˜600 to 900 L volume to provide a solution of compound F in ethyl acetate. The resulting ethyl acetate solution of compound F was carried forward directly to the next processing step.

Preparation of Compound G

Added 1460 L ethyl acetate to the previously prepared solution of compound F. The reaction mass was cooled to −65 to −70° C. Ozone was introduced subsurface to the reactor for 3.5 h HPLC until the reaction was judged complete by HPLC. The solution was purged of excess ozone and oxygen with nitrogen. A 5° C. solution of 113 kg triphenylphosphine (0.43 kmol, 1.20 eq) and 450 kg ethyl acetate was added over ˜10 minutes to the reaction mass with a 90 kg ethyl acetate chase. The reaction mass was warmed to 0° C. over 4 h. After 12 h at 0° C. the reaction mass was warmed to 12° C. and determined to be complete by HPLC. The reaction mass was washed with a solution of 13 kg sodium chloride in 980 L water. The resulting ethyl acetate solution of compound G was carried forward directly to the next processing step. The characterization data is from a sample of compound G purified by column chromatography. ¹H NMR (400 MHz, CDCl₃) δ9.75 (1H, s), 8.09 (1H, d, J=8.4 Hz), 7.90 (1H, d, J=8.4 Hz), 7.71 (1H, dd, J₁=8.3, J₂=8.3, Hz), 7.53 (1H, dd, J₁=8.3, J₂=8.3 Hz), 7.43 (1H, s), 7.38 (2H, d, J=9.2 Hz), 7.01 (2H, d, J=9.2 Hz), 6.18 (1H, s), 5.47 (2H, s), 4.25-4.09 (3H, m), 3.58 (1H, d, J=17.6 Hz), 2.74 (1H, s), 1.38 (9H, s), 1.18 (3H, t). ¹³C NMR (100 MHz, CD₃OD) δ199.3, 171.4, 158.9, 158.1, 154.0, 147.7, 141.8, 129.5, 129.3, 126.9, 126.1, 124.1, 122.7, 120.3, 115.0, 66.8, 62.5, 61.2, 28.2, 25.3, 13.8. MS (EI+): m/z: 493.2 (M+1), 437.1, 376.12, 350.1.

Preparation of Compound H

To the previously prepared solution of compound G in ethyl acetate was added 60.1 kg diisopropylethylamine (0.47 kmol, 1.3 eq) and 84.5 kg D-leucine methyl ester hydrochloride (0.47 kmol, 1.3 eq). The batch was cooled to 0 to 5° C. and held for 1 h. Portionwise added 110 kg sodium triacetoxyborohydride (0.52 kmol, 1.45 eq) at 0 to 5° C. Aged at 0±5° C. for 2 h at which point the reaction was judged complete by HPLC. Added 2000 L water and separated the phases. Added 150 kg toluene to the organic phase and extracted the organic phase with four portions (1000 L, 720 L, 720 L, 720 L) of an aqueous citric acid solution. Extracted a fifth time with a solution of 222 kg citric acid in 580 L water. The aqueous citric acid extracts were combined and washed with a mixture of 788 L ethyl acetate and 74 kg toluene. Added 550 L ethyl acetate and cooled the aqueous phase to 10 to 15° C. Adjusted the pH to 4.6 by addition of 866 L 30% aqueous sodium hydroxide while maintaining the batch temperature at <25° C. Separated the layers and back-extracted the aqueous phase with 790 L ethyl acetate. The two ethyl acetate phases were combined and washed with 500 L water to provide an ethyl acetate solution of a product of the above reductive amination of compound G. The ethyl acetate solution was distilled at atmospheric pressure to ˜800 L final volume (FIO: KF=0.166%). 400 L ethyl acetate was added and the batch again distilled to ˜800 L final volume. The reaction mass was held at reflux for 13 hours and the cyclization to the lactam compound H was judged complete by HPLC analysis. Solvent exchanged to 2-propyl alcohol by atmospheric distillation at a volume of 600 to 700 L by portionwise addition of 2300 L 2-propyl alcohol at atmospheric pressure. Distilled to a final volume of ˜350 L. Cooled to 0 to 5° C. and seeded with 0.5 kg compound H. The crystallization was aged for 24 hours at 0 to 5° C. The slurry was filtered on a centrifuge and the cake was washed with 250 L of 2-propyl alcohol. The product was dried at 40° C. for 8 h to afford 122 kg compound H, 59% overall yield from 120 kg compound E was obtained. ¹H NMR (400 MHz, CDCl₃) δ8.09 (1H, d, J=8.4 Hz), 7.91 (1H, d, J=8.4 Hz), 7.72 (1H, dd, J₁=8.3, , J₂=8.3, Hz), 7.53 (1H, dd, J₁=8.3, J₂=8.3, Hz), 7.48 (1H, s), 7.46 (2H, d, J=9.2 Hz), 7.00 (2H, d, J=9.2 Hz), 5.64 (1H, s), 5.49 (2H, s), 4.93 (1H, s, Br), 3.58 (3H, s), 3.40 (2H, m), 2.89 (1H, t), 2.77 (1H, s), 2.76 (3H, s), 1.81-1.72 (2H, m), 1.58 (1H, m), 1.42 (9H, s), 0.97 (6H, m). ¹³C NMR (100 MHz, CD₃OD) δ173.7, 171.4, 158.9, 157.9, 154.7, 147.7, 142.0, 133.2, 129.4, 127.3, 126.1, 124.1, 122.6, 120.3, 114.9, 66.8, 63.2, 52.4, 52.1, 40.5, 36.9, 28.3, 25.4, 24.6, 23.3, 21.1. MS (DCI/NH₃): m/z: 576.3 (M+1), 520.2, 459.1. Analysis Calculated for C₃₃H₄₁N₃O₆: C, 68.85; H, 7.18; N, 7.30. Found: C, 68.69; H, 7.06; N, 7.14.

Preparation of Compound I

To a 200 gallon glass-lined reactor was charged 30.2 kg Compound H (0.052 kmol) and 90 kg methyl alcohol. Added 5.8 kg methanesulfonic acid (MSA, 0.06 kmol, 1.15 eq) gradually at 25° C. The batch was heated slowly to 55° C. Added another 5.8 kg methanesulfonic acid (0.06 kmol, 1.15 eq) gradually over 30 min at 55° C. The reactor contents were aged at 55° C. for 2 h and sampled for reaction completion (HPLC criterion: <0.5 A % Compound H). Added 214 L 2-propyl alcohol at 55° C. Cooled to 15° C. and aged at 15° C. for 1 h. Isolated on a centrifuge and washed the cake with 2-propyl alcohol (3×50 kg). Dried under vacuum at 50° C. to provide 31.7 kg Compound I 90% yield. ¹H NMR (400 MHz, DMSO d₆) δ9.07 (4H, s), 8.51 (1H, d, J=8.4Hz), 8.32 (1H, d, J=8.6 Hz), 8.14 (1H, s), 8.14 (1H, t, J=8.6 Hz), 7.96 (1H, t, J=8.6 Hz), 7.61 (d, 2H, J=8.9 Hz), 7.39 (d, 2H, J=9.0 Hz), 5.94 (s, 2H), 4.80 (dd, 1H, J₁=4.0 Hz, J₂=11.3 Hz), 3.62 (s, 3H), 3.51 (1H, t, J=9.5 Hz), 3.15-3.28 (1H, m), 3.01 (3H, s,), 2.65-2.75 (1H, m), 2.48-2.60 (2H, m), 2.43 (6H, s), 1.85 (1H, t, J=12.2 Hz), 1.52-1.71 (2H, m), 0.96 (3H, d, J=6.3 Hz), 0.93 (3H, d, J=6.10 Hz) ¹³C NMR (100 MHz, CDCl₃) δ172.9, 172.5, 160.6, 159.7, 156.5, 139.1, 136.1, 131.2, 130.0, 128.9, 126.3, 126.0, 122.1, 117.2, 67.8, 64.3, 53.4, 50.1, 41.5, 40.1, 38.1, 33.8, 26.2, 24.0, 22.0, 21.5. Analysis Calculated for C₃₀H₄₁N₃O₁₀S₂: C, 53.96; H, 6.19; N, 6.29; S, 9.60. Found: C, 53.83; H, 5.97; N, 6.15; S, 9.76.

Preparation of Compound J

To a 30-gallon glass-lined reactor were charged 10 kg of hydroxylamine hydrochloride (0.14 kmol, 4.7 eq) and 15.6 kg methanol. The batch temperature was set to 50° C. and 64.6 kg of a 25 wt % solution of sodium methoxide in methanol was charged (sodium methoxide: 16.15 kg, 0.3 kmol, 10 eq) followed by a methanol rinse of the charging line. The reactor contents were heated to 55° C. and aged for 15 min. The batch was then cooled to 25° C. and filtered through a 36″ Nutsche filter using a polypropylene filter bag. The filtrate was collected in a 100-gallon glass-lined reactor and cooled to 10° C. (2R)-2-{(3R)-3-amino-3-[4-(2-methyl-quinolin-4-ylmethoxy)-phenyl]-2-oxo-pyrrolidin-1-yl}-4-methyl-pentanoic acid methyl ester bis-methanesulfonic acid salt (compound I)(20 kg, 0.03 kmol) was added and the batch was warmed to 25° C. for aged for 1 h. The reactor contents were sampled for reaction completion (HPLC criterion: >99.5 A % compound A). Once the reaction was deemed complete, ˜55 kg of 2N HCl solution (prepared using 126 kg purified water and 30 kg of concentrated HCl) was added and the reaction mass was sampled for pH measurement (acceptance criterion: pH ˜7.0). The batch was vacuum-distilled at ˜35° C. to remove ˜25 L methanol. The batch was then heated to 50° C. and a 1 L slurry of compound A (150 g) 1:4 methanol/water (volume ratio) was added. Water (92 L) was added uniformly over 1 hour at 50° C. to induce crystallization. The batch was cooled to 5° C. over a period of 2 h. The contents were filtered and the product washed first with a mixture of methanol/water (1:4 volume ratio) and then with pure water. The wet cake (˜20 kg) was analyzed to determine the weight % of water and charged to a clean 100-gallon reactor. Isopropyl alcohol (34 kg) was added and the batch was heated to 55° C. Once all the solids were dissolved, water was added to adjust the volume ratio to ˜55% water, 45% isopropyl alcohol. At 55° C., 2 L slurry of milled Compound A seeds (˜700 g) in 1:4 isopropyl alcohol/water (volume ratio) was charged. Purified water (67 kg) was charged through a cartridge filter gradually over a period of 3 h. The batch was cooled from 55° C. to 20° C. in 2 h, aged for 30 min and filtered through a 36″ Nutsche filter using a Dacron® filter bag. The filter-cake was washed three times with a mixture of isopropyl alcohol-water (1^{st} wash: 38 kg water, 8 kg isopropyl alcohol; 2^{nd} and 3^{rd} washes: 19 kg water, 4 kg isopropyl alcohol). The product was dried in a tray dryer under vacuum at 50° C. to provide 12.0 kg of Compound A in 84% yield.

### Example 2

### Preparation of Oral DPC 333 Formulations

Stock solutions of wetting agent, simple syrup:distilled water, and hydrochloric acid (HCl) are prepared within 24 hours of administration and stored at room temperature. The stock solution of the wetting agent, 0.5% (w/v) Pluronic® F68, in distilled water is prepared by adding 100 mL of distilled water to 500 mg of Pluronico® F68 and sonicating for ten minutes. A stock solution of simple syrup:distilled water in the ratio of 1:1.5 is prepared by adding 240 mL of the syrup, NF to 360 mL of distilled water and sonicating for five minutes until a homogeneous solution is obtained. A stock solution of hydrochloric acid is prepared by adding 4.3 mL of the hydrochloric acid (the 36%, w/w solution) to a 50-mL volumetric flask, adding a sufficient quantity of distilled water to a total volume of 50-mL, and sonicating for one minute.

Within six hours of administration, a stock solution of DPC 333 in hydrochloric acid is prepared as outlined below:

A bottle containing 225 mg of DPC 333 is tapped until all the powder flows freely. Hydrochloric acid stock solution (1 mL) is then added. The bottle is then vortexed until the powder has completely dissolved (approximately one minute).

Distilled water (14 mL) is then added and the bottle is shaken vigorously for a few seconds.

The active DPC formulation is prepared within two hours of preparing the stock solution using the following procedure:

For 15 mg dose, one milliliter of the stock solution of DPC 333 is transferred to an empty bottle. Two milliliters of the 0.5% Pluronic® F68 stock solution is then added to the bottle. The bottle is swirled until all the powder is wetted. Fifty-seven milliliters of the simple syrup:distilled water stock solution is then added to the bottle and shaken vigorously to mix.

For the 25 mg dose, 1.67 mL of the stock solution of DPC 333 is transferred into an empty bottle. Two milliliters of the 0.5% Pluronic® F68 stock solution is added to the bottle. The bottle is then swirled until all the powder is wetted. Fifty-six milliliters of the simple syrup:distilled water stock solution is added to the bottle and shaken vigorously to mix.

For the 40 mg dose, a bottle containing 40 mg of DPC 333 is tapped until all the powder flows freely. 2.2 mL of the 0.5% Pluronic® F68 stock solution is added to the bottle and the bottle is swirled until all the powder is wetted. 58 mL of the simple syrup:distilled water stock solution is then added to the bottle and shaken vigorously to mix.

For the 80 mg dose, two bottles each containing 40 mg of DPC 333 are tapped until all the powder flows freely. 2.2 mL of the 0.5% Pluronic® F68 stock solution is added to each bottle and the bottles are swirled until all the powder is wetted. 28 mL of the simple syrup:distilled water stock solution is added to each bottle and shaken vigorously to mix.

For the 120 mg dose, three bottles each containing 40 mg of DPC 333 are tapped until all the powder flows freely. 2.2 mL of the 0.5% Pluronic® F68 stock solution is added to each bottle and the bottles are swirled until all the powder is wetted. 18 mL of the simple syrup:distilled water stock solution is added to each bottle and shaken vigorously to mix.

For the 225 mg dose, a bottle containing 225 mg of DPC 333 is tapped until all the powder flows freely. 2.2 mL of the 0.5% Pluronic® F68 stock solution is added to the bottle and the bottle is swirled until all the powder is wetted. 58 mL of the simple syrup: distilled water stock solution is added to the bottle and shaken vigorously to mix.

For the 345 mg dose, one bottle containing 225 mg of DPC 333 and three bottles each containing 40 mg of DPC 333 are tapped until all the powder flows freely. 2.2 mL of the 0.5% Pluronic® F68 stock solution is added to each bottle and the bottles are swirled until all the powder is wetted. 13 mL of the simple syrup:distilled water stock solution is added to each bottle and shaken vigorously to mix.

For the 530 mg dose, two bottles each containing 225 mg of DPC 333 and two bottles each containing 40 mg of DPC 333 are tapped until all the powder flows freely. 2.2 mL of the 0.5% Pluronic® F68 stock solution is added to each bottle and the bottles are swirled until all the powder is wetted. 13 mL of the simple syrup:distilled water stock solution is added to each bottle and shaken vigorously to mix.

### Example 3

### Administration

The active DPC formulation must be administered within four hours of preparation. Just prior to administration, the bottle or bottles are shaken vigorously for 30 seconds and administered immediately to the subject. Following administration, distilled water is added to the bottle or bottles, shaken gently for a few seconds and then administered immediately to the subject. This step is repeated twice so that each subject receives a total volume of approximately 240 mL (dose+distilled water rinses).

What is claimed is:

1. An oral dosage form of DPC 333, said DPC 333 dosage form providing, when administered as a single dose ranging from 15 mg to 530 mg to a subject,
a mean terminal disposition half-life (T_{1/2}) of about 2 hours to about 7 hours.

2. An oral dosage form of DPC 333, said DPC 333 dosage form providing, when administered as a single dose greater than 225 mg to a subject,
a greater than proportional increase in mean area under the plasma concentration versus time curve from time zero to time infinity (AUC).

3. An oral dosage form of DPC 333, said DPC 333 dosage form providing, when administered with food, a decreased AUC of about 23% and a decreased Cmax of about 40% as compared said dosage form when administered in a fasted state.

4. An oral dosage form of DPC 333, said DPC 333 dosage form providing:
when administered as a single dose of 15 mg, a mean maximum plasma concentration (Cmax) of about 88 nM to about 180 nM, a mean time of maximum plasma concentration (Tmax) of about 0.5 hours to about 1.0 hours, or a mean area under the plasma concentration versus time curve from time zero to time infinity (AUC) of about 255 nM▪hour to about 430 nM▪hour,;
when administered as a single dose of 25 mg, a mean maximum plasma concentration (Cmax) of about 170 nM to about 435 nM, a mean time of maximum plasma concentration (Tmax) of about 0.5 hours to about 1.0 hours, or a mean area under the plasma concentration versus time curve from time zero to time infinity (AUC) of about 450 nM▪hour to about 900 nM▪hour;
when administered as a single dose of 40 mg, a mean maximum plasma concentration (Cmax) of about 355 nM to about 655 μM, a mean time of maximum plasma concentration (Tmax) of about 0.25 hours to about 1.0 hours, or a mean area under the plasma concentration versus time curve from time zero to time infinity (AUC) of about 930 nM▪hour to about 1425 nM▪hour;
when administered as a single dose of 80 mg, a mean maximum plasma concentration (Cmax) of about 965 nM to about 1870 μM, a mean time of maximum plasma concentration (Tmax) of about 0.5 hours to about 1.0 hours, or a mean area under the plasma concentration versus time curve from time zero to time infinity (AUC) of about 2245 nM▪hour to about 4160 nM▪hour;
when administered as a single dose of 120 mg, a mean maximum plasma concentration (Cmax) of about 1535 nM to about 2345 nM, a mean time of maximum plasma concentration (Tmax) of about 0.5 hours to about 1.0 hours, or a mean area under the plasma concentration versus time curve from time zero to time infinity (AUC) of about 3335 nM▪hour to about 6210 nM▪hour;
when administered as a single dose of 225 mg, a mean maximum plasma concentration (Cmax) of about 2625 nM to about 3650 μM, a mean time of maximum plasma concentration (Tmax) of about 0.5 hours to about 1.0 hours, or a mean area under the plasma concentration versus time curve from time zero to time infinity (AUC) of about 6380 nM▪hour to about 9400 nM▪hour;
when administered as a single dose of 345 mg, a mean maximum plasma concentration (Cmax) of about 6585 nM to about 15420 nM, a mean time of maximum plasma concentration (Tmax) of about 0.25 hours to about 1.0 hours, or a mean area under the plasma concentration versus time curve from time zero to time infinity (AUC) of about 18375 nM▪hour to about 28820 nM▪hour; or
when administered as a single dose of 530 mg, a mean maximum plasma concentration (Cmax) of about 9450 nM to about 18072 nM, a mean time of maximum plasma concentration (Tmax) of about 0.5 hours to about 1.0 hours, or a mean area under the plasma concentration versus time curve from time zero to time infinity (AUC) of about 22040 nM▪hour to about 39480 nM▪hour.

4. A method of inhibiting human tumor necrosis factor-alpha convertase (TACE) comprising administering to a mammal the oral dosage form of DPC 333 of claim 1.

5. A method of treating inflammatory diseases characterized by TNFα overproduction comprising administering to a mammal the oral dosage form of DPC 333 of claim 1.

6. The method of claim 5 wherein the inflammatory disease is rheumatoid arthritis.

7. A method of treating a condition or disease mediated by MMPs, TACE, aggrecanase, or a combination thereof in a mammal, comprising: administering to the mammal in need of such treatment the oral dosage form of DPC 333 of claim 1.

8. A method comprising: administering the oral dosage form of DPC 333 of claim 1 to treat a condition or disease mediated by MMPs, TACE, aggrecanase, or a combination thereof.

9. A method of treating a disease or condition according to claim 7, wherein the disease or condition is selected from acute infection, acute phase response, age related macular degeneration, alcoholic liver disease, allergy, allergic asthma, anorexia, aneurism, aortic aneurism, asthma, atherosclerosis, atopic dermatitis, autoimmune disease, autoimmune hepatitis, Bechet's disease, cachexia, calcium pyrophosphate dihydrate deposition disease, cardiovascular effects, chronic fatigue syndrome, chronic obstruction pulmonary disease, coagulation, congestive heart failure, corneal ulceration, Crohn's disease, enteropathic arthropathy, Felty's syndrome, fever, fibromyalgia syndrome, fibrotic disease, gingivitis, glucocorticoid withdrawal syndrome, gout, graft versus host disease, hemorrhage, HIV infection, hyperoxic alveolar injury, infectious arthritis, inflammation, intermittent hydrarthrosis, Lyme disease, meningitis, multiple sclerosis, myasthenia gravis, mycobacterial infection, neovascular glaucoma, osteoarthritis, pelvic inflammatory disease, periodontitis, polymyositis/dermatomyositis, post-ischaemic reperfusion injury, post-radiation asthenia, psoriasis, psoriatic arthritis, pulmonary emphysema, pydoderma gangrenosum, relapsing polychondritis, Reiter's syndrome, rheumatic fever, rheumatoid arthritis, sarcoidosis, scleroderma, sepsis syndrome, Still's disease, shock, Sjogren's syndrome, skin inflammatory diseases, solid tumor growth and tumor invasion by secondary metastases, spondylitis, stroke, systemic lupus erythematosus, ulcerative colitis, uveitis, vasculitis, and Wegener's granulomatosis.

10. A method for treating an inflammatory disorder, comprising: administering, in combination, to a host in need thereof, a therapeutically effective amount of
(a) the oral dosage form of DPC 333 of claim 1; and,
(b) one or more additional anti-inflammatory agents selected from selective COX-2 inhibitors, interleukin-1 antagonists, dihydroorotate synthase inhibitors, p38 MAP kinase inhibitors, TNF-α inhibitors, TNF-α sequestration agents, and methotrexate.

11. An article of manufacture, comprising:
(a) a first container;
(b) a pharmaceutical composition located within the first container, wherein the composition, comprises: the oral dosage form of DPC 333 of claim 1; and,
(c) a package insert stating that the pharmaceutical composition can be used for the treatment of an inflammatory disorder.

12. An article of manufacture according to claim 11, further comprising:
(d) a second container;
wherein components (a) and (b) are located within the second container and component (c) is located within or outside of the second container.

13. An article of manufacture, comprising:
(a) a first container;
(b) a pharmaceutical composition located within the first container, wherein the composition, comprises: the oral dosage form of DPC 333 of claim 1; and,
(c) a package insert stating that the pharmaceutical composition can be used in combination with a second therapeutic agent to treat an inflammatory disorder.

14. An article of manufacture according to claim 13, further comprising:
(d) a second container;
wherein components (a) and (b) are located within the second container and component (c) is located within or outside of the second container.
